(19) United States
(12) Patent Application Publication
(10) Pub. No.: US 2006/0069428 A1
(43) Pub. Date: Mar. 30, 2006

(54) Thin film medical device and delivery system

(76) Inventor: Frederick Feller III, Coral Springs, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/231,088

(22) Filed: Sep. 20, 2005

Related U.S. Application Data
(60) Provisional application No. 60/611,485, filed on Sep. 20, 2004.

(51) Int. Cl. A61F 2/06

(52) U.S. Cl. 623/1.44

(57) ABSTRACT

The present invention relates to an intraluminal thin film medical device particularly well suited for occlusion of an aneurysm, vessel side branch or dissection of a body lumen or duct, such as an artery or vein. The medical device has a thin film tube capable of being longitudinally stretched by the application of mechanical energy to achieve a smaller circumferential profile, and self-expand to the pre-stretched length and diameter upon release of the mechanical energy. To assist the thin film during expansion a plurality of slots are incised in the tube wall. The slots open and assist the thin film tube to longitudinally stretch, and substantially close when the thin film tube self-expands to the pre-stretched length and diameter.

### FIELD OF THE INVENTION

The present invention relates to a thin film medical device, and in particular to an intraluminal thin film medical device and delivery system. This medical device and delivery system are particularly well suited for occlusion of an aneurysm, vessel side branch or dissection of a body lumen or duct, such as an artery or vein.

### BACKGROUND OF THE INVENTION

There are many instances when it may be desirable to permanently occlude a vessel in the human body. Examples of when permanent occlusion of a vessel might be desirable include: occlusion of an aneurysm or side branch vessel; therapeutic occlusion, or embolization, of the renal artery; occlusion of a Blalock-Taussig Shunt; pulmonary arteriovenous fistulae and transjugular intrahepatic stent shunt occlusion; some non-vascular applications, such as therapeutic ureteric occlusion; and the occlusion of vessels feeding large cancerous tumors.

In the past, certain coiled stents, stent grafts or detachable balloons have been utilized for providing permanent occlusion of vessels. Stent-grafts are essentially endoluminal stents with a discrete covering on either or both of the luminal and abluminal surfaces of the stent that occludes the open spaces, or interstices, between adjacent structural members of the endoluminal stent. It is known in the art to fabricate stent-grafts by covering the stent with endogenous vein or a synthetic material, such as woven polyester known as DACRON, or with expanded polytetrafluoroethylene. Additionally, it is known in the art to cover the stent with a biological material, such as a xenograft or collagen.

There are certain problems associated with coiled stents, including, migration of the coiled stent within the vessel to be occluded, perforation of the vessel by the coiled stent, and failure to completely thrombose, or occlude, the vessel. Another disadvantage associated with such coiled stents is that the vessel may not be immediately occluded following placement in the vessel. Disadvantages associated with detachable occlusion balloons include premature detachment with distal embolization, or occlusion, and they are believed to require a longer period of time for the user of the device to learn how to properly use such detachable occlusion balloons.

In addition to vessel occlusion, conventional graft type intraluminal medical devices are frequently used post-angioplasty in order to provide a structural support for a blood vessel and reduce the incidence of restenosis following percutaneous balloon angioplasty. A principal example are endovascular stents which are introduced to a site of disease or trauma within the body's vasculature from an introductory location remote from the disease or trauma site using an introductory catheter, passed through the vasculature communicating between the remote introductory location and the disease or trauma site, and released from the introductory catheter at the disease or trauma site to maintain patency of the blood vessel at the site of disease or trauma. Stent-grafts are delivered and deployed under similar circumstances and are utilized to maintain patency of an anatomic passageway, for example, by reducing restenosis following angioplasty, or when used to exclude an aneurysm, such as in aortic aneurysm exclusion applications.

While these medical devices have specific advantages, their overall size, in particular the diameter and delivery profile, are significant disadvantages that render these devices prohibitive for certain uses. Another significant disadvantage is the limited flexibility these devices have for navigating paths through small and/or tortuous vessels. As such, they may not be desirable for many small diameter vessel applications, for example neurovascular vessels.

What is needed is a medical device capable of occluding various parts of a vessel that can assume a reduced diameter and delivery profile.

### SUMMARY OF THE INVENTION

The present invention relates to an intraluminal thin film medical device particularly well suited for occlusion of an aneurysm, vessel side branch or dissection of a body lumen or duct, such as an artery or vein. In one embodiment of the invention, the medical device comprises a thin film tube capable of being longitudinally stretched by the application of mechanical energy to achieve a smaller circumferential profile. Once the mechanical energy is released, the thin film tube is capable of self-expanding to the pre-stretched length and diameter. The medical device further comprises a plurality of slots incised in the tube wall. The slots are arranges such that they open and assist the thin film tube to longitudinally stretch, and substantially close when the thin film tube self-expands to the pre-stretched length and diameter.

Another embodiment of the present medical device for occluding a body vessel comprises a thin film tube capable of being longitudinally stretched by the application of mechanical energy to achieve a smaller circumferential profile, and self-expand to the pre-stretched length and diameter upon release of the mechanical energy. A plurality of apertures are incised in the thin film tube wall such the apertures assist the thin film tube to longitudinally stretch.

Still another embodiment of the medical device for occluding a body vessel comprises a thin film tube capable of being longitudinally stretched by the application of mechanical energy to achieve a smaller circumferential profile, and self-expand to the pre-stretched length and diameter upon release of the mechanical energy. The medical device further comprises a stent attached to the interior surface of the thin metallic film.

### BRIEF DESCRIPTION OF THE DRAWINGS

FIG. 1A show a perspective view of medical device fabricated from a thin film tube in the deployed or “pre-stretched” configuration according to one embodiment of the present invention.

FIG. 1B shows a perspective view of a medical device fabricated from a thin film tube in the stretched reduced profile and restrained position according to one embodiment of the present invention.

FIG. 1C illustrates a perspective view of a medical device according to one embodiment of the present invention where only a portion of the radial slots along the proximal end and distal end are open, while the radial slots in the intermediate section remain substantially closed.

FIG. 2 is a perspective partial section view showing a medical device deployed in a vessel according to one embodiment of the present invention.

FIG. 3A is a perspective partial section view showing a medical device according to an embodiment of the present invention deployed over an aneurysm in a vessel wall, where the medical device has a proximal stent attaching the thin film tube to the vessel wall.

FIG. 3B is a perspective partial section view showing a medical device according to an embodiment of the present invention deployed over an aneurysm in a vessel wall, where the medical device has a proximal stent attaching the thin film tube to the vessel wall along the proximal end, as well as a distal stent attaching the distal end of the thin film tube to the vessel wall along the distal end.

FIG. 3C is a perspective partial section view showing a medical device according to an embodiment of the present invention deployed over an aneurysm in a vessel wall, where the medical device has a stent structure having multiple hoop sections arranged axially along a central longitudinal axis.

FIG. 4 is a longitudinal section view illustrating a medical device having a self-supporting metallic thin film tube loaded on a delivery catheter according to one embodiment of the present invention.

FIG. 5 is a longitudinal section view illustrating a medical device having a self-expanding stent for additional radial support according to one embodiment of the present invention.

FIG. 6 is a longitudinal section view illustrating a medical device having a balloon expandable stent for additional radial support according to one embodiment of the present invention.

### DETAILED DESCRIPTION OF A PREFERRED EMBODIMENT

The present invention discloses a thin film medical device particularly well suited for occlusion of an aneurysm or vessel side branch, or dissection of body lumen or duct, such as an artery or vein. One advantage of the present invention is that is provides a biocompatible graft material that enables a less invasive delivery of the medical device to a vascular site for occluding blood flow while sill allowing blood flow through the main vessel at the implant location.

Although this specification provides detailed description for implantation of the medical device in a artery or vein, one of skill in the art would understand that modifications of the disclosed invention would also be well suited for use on other body lumens and anatomical passageways, such as, for example those found in the cardiovascular, lymphatic, endocrine, renal, gastrointestinal and or reproductive systems.

The primary component of the present invention is a thin film made primarily of a substantially self-supporting biocompatible metal or psuedometal. The thin film may be fabricated either as single layer, or a plurality of layers. The terms “thin film”, “metal film”, “thin metallic film”, and “metallic thin film” are used synonymously in this application to refer to a single or plural layer film fabricated of biocompatible metal or biocompatible pseudometals having a thickness greater than 0.1 μm but less than 250 μm, preferably between 1 and 50 μm. In some particular embodiments of the invention, such as where the thin film is used as a structural support component, the thin film may have a thickness greater than approximately 25 μm. In other embodiments, for example, where the thin film is used as a cover member with additional structural support, the thin film may have a thickness of between approximately 0.1 μm and 30 μm, most preferably between 0.1 μm and 10 μm.

In a preferred embodiment, the medical device is fabricated from a shape memory thin metallic film or pseudometallic film having super elastic characteristics. One example of a shape memory metallic thin film is Nickel Titanium (Nitinol) formed into a tubular structure.

Nitinol is utilized in a wide variety of applications, including medical device applications as described above. Nitinol or NiTi alloys are widely utilized in the fabrication or construction of medical devices for a number of reasons, including its biomechanical compatibility, its bio-compatibility, its fatigue resistance, its kink resistance, its uniform plastic deformation, its magnetic resonance imaging compatibility, its ability to exert constant and gentle outward pressure, its dynamic interference, its thermal deployment capability, its elastic deployment capability, its hysteresis characteristics, and is moderately radiopacity.

Nitinol, as described above, exhibits shape memory and/or super elastic characteristics. Shape memory characteristics may be simplistically described as follows. A metallic structure, for example, a Nitinol tube that is in an Austenitic phase may be cooled to a temperature such that it is in the Martensitic phase. Once in the Martensitic phase, the Nitinol tube may be deformed into a particular configuration or shape by the application of stress. As long as the Nitinol tube is maintained in the Martensitic phase, the Nitinol tube will remain in its deformed shape. If the Nitinol tube is heated to a temperature sufficient to cause the Nitinol tube to reach the Austenitic phase, the Nitinol tube will return to its original or programmed shape. The original shape is programmed to be a particular shape by well-known techniques as briefly described above.

Super elastic characteristics may be simplistically described as follows. A metallic structure for example, a Nitinol tube that is in an Austenitic phase may be deformed to a particular shape or configuration by the application of mechanical energy. The application of mechanical energy causes a stress induced Martensitic phase transformation. In other words, the mechanical energy causes the Nitinol tube to transform from the Austenitic phase to the Martensitic phase. By utilizing the appropriate measuring instruments, one can determined that the stress from the mechanical energy causes a temperature drop in the Nitinol tube. Once the mechanical energy or stress is released, the Nitinol tube undergoes another mechanical phase transformation back to the Austenitic phase and thus its original or programmed shape. As described above, the original shape is programmed by well know techniques. The Martensitic and Austenitic phases are common phases in many metals.

Medical devices constructed from Nitinol are typically utilized in both the Martensitic phase and/or the Austenitic phase. The Martensitic phase is the low temperature phase. A material is in the Martensitic phase is typically very soft and malleable. These properties make it easier to shape or configure the Nitinol into complicated or complex structures. The Austenitic phase is the high temperature phase. A material in the Austenitic phase is generally much stronger than the materiel in the Martensitic phase. Typically, many medical devices are cooled to the Martensitic phase for manipulation and loading into delivery systems. When the device is deployed at body temperature, they return to the Austenitic phase.

Although Nitinol is described in this embodiment, it should not be understood to limit the scope of the invention. One of skill in the art would understand that other materials, both metallic and pseudo-metallic exhibiting similar shape memory and super-elastic characteristics may be used.

The tubular thin film structure is sized to match or be slightly greater than the diameter of the inner lumen of the body vessel when the tube is in the unrestrained (“self-expanded”) configuration. The inherent properties of the thin Nitinol tube are such that the tube is capable of being longitudinally stretched, which decreases the tube's diameter. Reducing the diameter allows the medical device to maintain a compact profile for insertion into a body lumen via a catheter during a percutaneous, endoluminal procedure. Accordingly, the inherent shape memory and super-elastic characteristics allow the thin metallic tube to be stretched and restrained in a reduced profile configuration, and then self-expand back to its original “pre-stretched” diameter once the restraint is removed. As the tube diametrically expands, it longitudinally contracts or foreshortens to its pre-stretched length and diameter.

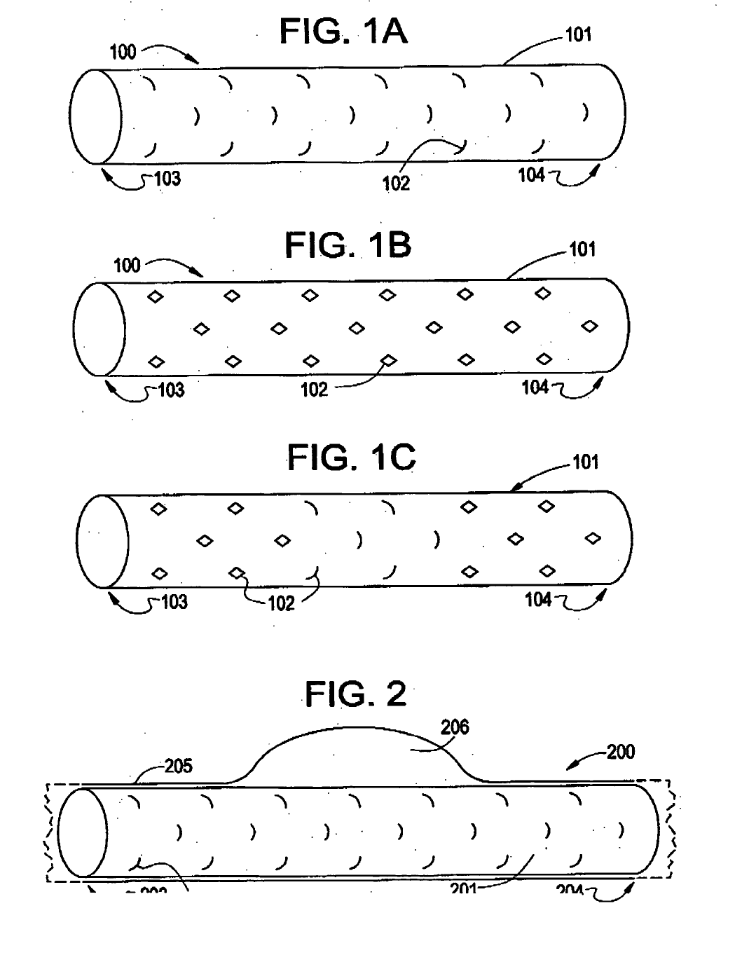

FIGS. 1A and 1B show a medical device fabricated from a Nitinol thin film tube according to one embodiment of the present invention. FIG. 1A shows the thin film medical device 100 in the deployed or “pre-stretched” configuration, while FIG. 1B shows the thin film medical device 100 in the stretched reduced profile and restrained position.

To facilitate the ability for the thin film medical device 100 to stretch in the longitudinal direction, the tubular structure 101 has a plurality of radial slots 102 incised or formed circumferentially through the tube 101 wall. In one embodiment, the slots are in the form of slits made completely through the thin film tube wall 101. Alternatively, where the thin film is manufactured in layers, the radial slots 102 may be through one or more layers of the thin film tube 101 wall. As the thin film tube 101 is longitudinally stretched, the slots 102 open, creating an opening in the tube 101 wall. When the thin film tube 101 is allowed to return to the pre-stretched (radially expanded) configuration, the radial slots 102 close, excluding blood flow in the circumferential direction.

The terms exclude, excluding and variations thereof, should not be construed as having zero porosity and completely preventing fluid flow. Instead, the closed slits and apertures in the thin film that exclude fluid flow may have openings that are small enough to substantially occlude blood flow through the thin film tube 101 wall. A medical device 100 illustrating all the radial slots 102 in the open position is illustrated in FIG. 1B.

The medical device 100 may also be designed so that some of the radial slots 102 can open, while other radial slots 102 remain substantially closed. FIG. 1C illustrates a medical device 100 where only a portion of the radial slots 102 along the proximal end 103 and distal end 104 are open, while the radial slots 102 in the intermediate section remain closed.

In another embodiment of the present invention, the medical device 100 may also has apertures 102 incised or formed through the tube wall in various shapes. The shapes may be chosen to facilitate longitudinal stretching and/or radial expansion of the thin film tube. Essentially, the apertures 102 in the thin film have longitudinal and latitudinal dimensions, thereby forming an opening in the thin film having a net free open area.

The above-described medical device 100 can be used, for example, across an aneurysm, side-branch vessel, or any vessel wall defect to exclude blood flow. In one embodiment of the invention, the tubular thin film 101 may be fabricated to a thickness that can support itself circumferentially. Alternatively, thinner films could be supported by a balloon or self-expanding stent or stents if additional radial support is needed.

FIG. 2 is a perspective partial section view showing a medical device 200 deployed in a vessel 205 according to one embodiment of the present invention. The vessel 205 has a weakened vessel wall causing an aneurysm 206, and the medical device 200 is deployed over the aneurysm 206. The medical device 200 is self-supporting, and does not require additional stent(s) for support. As described earlier, the medical device 200 comprises a thin metallic film tube 201 having a proximal end 203 and a distal end 204. The thin film tube 201 has a series of radial slots 202 arranged circumferentially along the thin film tube 201 longitudinal axis. Upon deployment from a catheter system, the radial slots 202 incised in the thin film tube 201 substantially close, excluding blood flow in the circumferential direction. This relieves pressure in the aneurysm 206, and mitigates potential medical conditions associated with the aneurysm 206 bursting. Reducing the pressure in the aneurysm 206 may also allow the vessel 205 wall to contract.

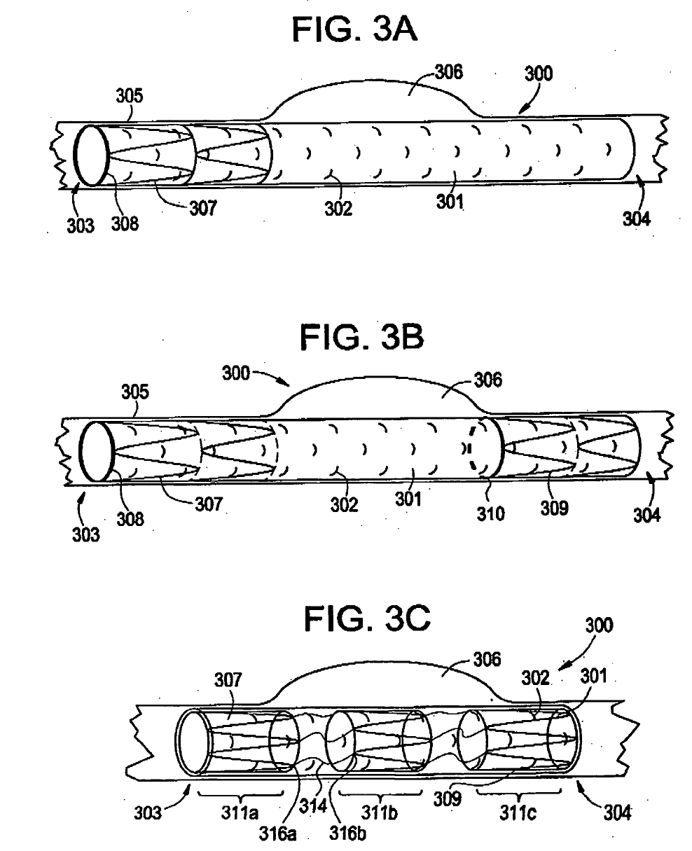

The medical device may also include one or more stents to assist in securing the thin film tube into the vessel wall. FIG. 3A shows a medical device 300 according to another embodiment of the present invention deployed over an aneurysm 306 in a vessel wall 305. Similar to the medical devices described above, the medical device 300 comprises a thin metallic film formed into a tube 301, having a proximal end 303 and distal end 304. The thin film tube 301 has a series of radial slots 302 incised circumferentially through the tube 301 wall. The medical device 300 additionally comprises a stent 307 along the proximal end 303.

The stent 307 disclosed comprises at least one hoop structure extending between the stent 307 proximal and distal ends, 303, 304 respectively. The hoop structure includes a plurality of longitudinally arranged strut members and a plurality of loop members connecting adjacent struts. Adjacent struts are connected at opposite ends in a substantially S or Z shaped sinusoidal pattern so as to form a plurality of cells. However, one of ordinary skill in the art would recognize that the pattern shaped by the struts is not a limiting factor, and other shaped patterns or radially expandable structures may be used.

As previously described, the stent 307 assists in anchoring the medical device 300 to the vessel 305 wall. The thin film tube 301 may be affixed to the stent 307 at anchor point 308. Attachment may be by any suitable attachment means, including adhesion resulting from radial pressure of the stent 307 against the thin metallic film tube 301, adhesion by means of a binder, heat, or chemical bond, and/or adhesion by mechanical means, such as welding or suturing between the stent 307 and the thin metallic film tube 301. It should be noted that the stent 307 does not necessarily have to be fixedly attached to the metallic film tube 301. Instead, the radially outward force that stent 307 exerts against the vessel wall may be adequate to hold the metallic thin film 301 in place.

In an alternate embodiment, the thin metallic film tube 301 may be anchored to the vessel 305 wall by a plurality of anchors. FIG. 3B shows a medical device 300 having a proximal stent 307 attaching the thin film tube 301 to the vessel 305 wall along the proximal end 303, as well as a distal stent 309 attaching the distal end of the thin film tube 301 to the vessel 305 wall along the distal end 304. Still one of skill in the art would understand that additional stents may be used to anchor the medical device 300 to the vessel 305 wall, such as additional proximal or distal anchors placed longitudinally along the thin film tube 301.

In a further alternate embodiment, stents having multiple hoop structures or longer hoop structures may be used to fully support the thin metallic film along all or substantially all of the film's length. FIG. 3C shows a medical device 300 having a multi-hoop stent 307 supporting the metallic thin film 301 substantially along the entire length of the thin metallic film 301.

The multiple hoop stent 307 illustrated in FIG. 3C comprises three hoop structures 311A through 311C connected by a plurality of bridge members 314. Each bridge member 314 comprises two ends 316A, 316B. One end 316A, 316B of each bridge 314 is attached to one hoop. Using hoop sections 311A and 311B for example, each bridge member 314 is connected at end 316A to the proximal end of hoop 311A, and at end 316B the distal end of hoop section 311B.

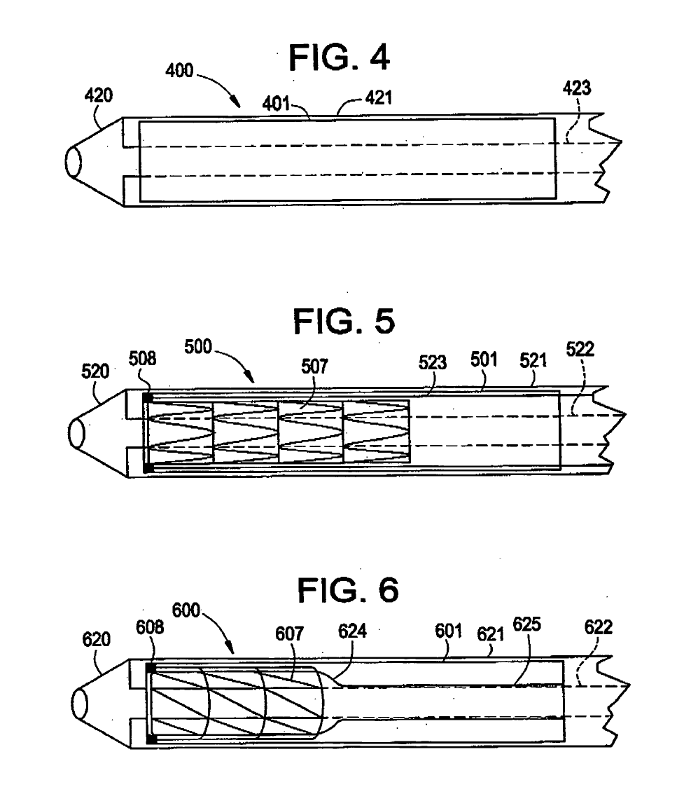

The various embodiments of the medical device described above are preferably delivered to the target area and subsequently deployed by a catheter system. FIG. 4 is a longitudinal section view illustrating a medical device 400 having a self-supporting metallic thin film tube 401 loaded on a delivery catheter 420 according to one embodiment of the present invention. The catheter 420 comprises an outer sheath 421 and an inner lumen 422. The outer sheath 421 serves to hold the thin film tube 401 in the longitudinally stretched position. The inner lumen 422 is substantially coaxial to the outer sheath 421 and provides a conduit for a guide wire.

To be deployed, the medical device 400 is mounted on the delivery catheter 420. A guide wire (not shown) is steered to the target area through well know means, and the delivery catheter 420/medical device 400 is loaded onto the guide wire using inner lumen 422. The catheter 420/medical device 400 is then pushed over the guide wire to the target site. Once properly located, the outer sheath 421 is retracted, allowing the thin film tube 401 to expand and longitudinally foreshorten to its unconstrained diameter. As previously described, this will allow the slots 402 (not shown) incised through the thin film tube 401 wall to substantially close and eliminate blood flow to the vessel wall defects.

The illustrated embodiment describes an over-the-wire delivery catheter. However, one of skill in the art would understand that other types of delivery catheters may also be used, include catheter utilizing a monorail design as are known in the art.

As previously described, very thin films may require extra radial support to adequately anchor the thin film in the vessel. In one embodiment, extra radial support could be supplied by radially expandable devices, such as radially expandable stents. FIG. 5 is a longitudinal section view illustrating a medical device 500 having a self-expanding stent 507 for additional radial support according to one embodiment of the present invention.

The catheter 520 for restraining and delivering the medical device 500 having a self-expanding stent 507 has three main components. Similar to the embodiment described above, the catheter 520 comprises an outer sheath 521 that serves to hold the thin film tube 501 in the longitudinally stretched position. Coaxial to the outer sheath 521 is a secondary sheath 523 of smaller diameter that serves to hold the self-expanding stent in a constrained position. As earlier described, the medical device 500 may have more than one stent for added radial support, i.e. may have stent 507 and 509 (not shown) as earlier described. In each case, secondary sheath 523 may serve to hold each radially expandable stent in the constrained position.

The third component of the medical device 500 is an inner lumen 522. The inner lumen 522 is substantially coaxial to the outer sheath 521 and the secondary sheath 523, and provides a conduit for a guide wire. The thin film tube 501 is affixed to the stent 507 at anchor point 508. As earlier described, attachment may be by any suitable attachment means, including adhesion resulting from radial pressure of the stent 507 against the thin metallic film tube 501, adhesion by means of a binder, heat, or chemical bond, and/or adhesion by mechanical means, such as welding or suturing between the stent 507 and the thin metallic film tube 501.

To be deployed, the medical device 500 is mounted on the delivery catheter 520. A guide wire (not shown) is steered to the target area through well-known means, and the delivery catheter 520/medical device 500 is loaded onto the guide wire using inner lumen 522. Alternatively, the delivery catheter 520/medical device 500 may be loaded onto the guide wire in a monorail fashion as is known in the art. The catheter 520/medical device 500 is then pushed over the guide wire to the target site. Once properly located, the outer sheath 521 is retracted, first allowing the thin film tube 501 to expand and longitudinally foreshorten to its unconstrained diameter. As previously described, this will allow the slots 502 (not shown) incised through the thin film tube 501 wall to substantially close and exclude blood flow to the vessel wall defects. The secondary sheath 523 may then be retracted, allowing the stent 507, and any other stents (not shown) to self-expand into the vessel wall (not shown). The radial pressure exerted by the stent 507 into the vessel wall anchors the stent 507 in place. As a result, the thin film tube 501 is further supported and anchored to the vessel wall.

In an alternate embodiment, the self-expanding stent may be replace with a balloon expandable stent. FIG. 6 is a longitudinal section view illustrating a medical device 600 having a balloon expandable stent 607 for additional radial support according to one embodiment of the present invention.

The catheter 620 for restraining and delivering the medical device 600 having a balloon expandable stent 607 has three main components. Similar to the embodiment described above, the catheter 620 comprises an outer sheath 621 that serves to hold the thin film tube 601 in the longitudinally stretched position. Coaxial to the outer sheath 621 is balloon catheter 625 having a balloon 624 mounted thereto. The balloon expandable stent 607 is mounted or crimped in a low profile configuration to the balloon catheter 625 over the expansion balloon 624. As earlier described, the medical device 600 may have more than one stent for added radial support, i.e. may have stent 607 and 609 (not shown), and possible others, as earlier described. In each case, each balloon 624 or balloons 624, on the balloon catheter 625 may serve to hold and deliver each radially expandable stent in the constrained position.

The third component of the medical device 600 is an inner lumen 622. The inner lumen 622 is substantially coaxial to the outer sheath 621 and the balloon catheter 625, and provides a conduit for a guide wire. In a preferred embodiment, the inner lumen 622 is an integral part of the balloon catheter 625. Alternatively, the catheter 620 may be a loop or similar capture device along the distal end to accept the guide wire in a monorail fashion. Monorail type catheters are known in the art.

The thin film tube 601 is preferably affixed to the stent 607 at anchor point 608. As earlier described, attachment may be by any suitable attachment means, including adhesion resulting from radial pressure of the stent 607 against the thin metallic film tube 601, adhesion by means of a binder, heat, or chemical bond, and/or adhesion by mechanical means, such as welding or suturing between the stent 607 and the thin metallic film tube 601.

To be deployed, the medical device 600 is mounted on the balloon catheter 625. A guide wire (not shown) is steered to the target area through well know means, and the balloon catheter 625/medical device 600 is loaded onto the guide wire using inner lumen 622. The catheter 625/medical device 500 is then pushed over the guide wire to the target site. Once properly located, the outer sheath 621 is retracted, first allowing the thin film tube 601 to expand and longitudinally foreshorten to its unconstrained diameter. As previously described, this will allow the slots 602 (not shown) incised through the thin film tube 601 wall to close and exclude blood flow to the vessel wall defects. The balloon 624 is then inflated (expanded), expanding the stent 607, and any other stents (not shown) into the vessel wall (not shown). The radial pressure exerted by the stent 607 into the vessel wall anchors the stent 607 in place. As a result, the thin film tube 601 is further supported and anchored to the vessel wall.

While a number of variations of the invention have been shown and described in detail, other modifications and methods of use contemplated within the scope of this invention will be readily apparent to those of skill in the art based upon this disclosure. It is contemplated that various combinations or sub combinations of the specific embodiments may be made and still fall within the scope of the invention. Moreover, all assemblies described are believed useful when modified to treat other vessels or lumens in the body, in particular other regions of the body where fluid flow in a body vessel or lumen needs to be excluded or regulated. This may include, for example, the coronary, vascular, non-vascular and peripheral vessels and ducts. Accordingly, it should be understood that various applications, modifications and substitutions may be made of equivalents without departing from the spirit of the invention or the scope of the following claims.

The following claims are provided to illustrate examples of some beneficial aspects of the subject matter disclosed herein which are within the scope of the present invention.

What is claimed is:

1. A medical device for occluding a body vessel comprising:
a thin film tube capable of being longitudinally stretched by the application of mechanical energy to achieve a smaller circumferential profile, and self-expand to the pre-stretched length and diameter upon release of the mechanical energy; and
a plurality of slots incised in the tube wall such the slots open and assist the thin film tube to longitudinally stretch, and substantially close when the thin film tube self-expands to the pre-stretched length and diameter.

2. The medical device of claim 1 wherein the thin film tube is made from a metallic thin film exhibiting super-elastic characteristics.

3. The medical device of claim 2 wherein the metallic thin film is made from a Nickel Titanium alloy.

4. The medical device of claim 1 wherein the thin film tube is made from a psuedometalic thin film exhibiting super-elastic characteristics.

5. The medical device of claim 1 wherein the thin film tube is self-supporting.

6. The medical device of claim 1 wherein the thin film tube is fabricated as a single layer of material.

7. The medical device of claim 1 wherein the thin film tube is fabricated as a plurality of layers of material.

8. The medical device of claim 1 wherein the slots are incised completely through the tube wall thickness.

9. The medical device of claim 1 wherein the slots are incised partially through the tube wall thickness.

10. The medical device of claim 1 further comprising a plurality of apertures in the tube wall.

11. The medical device of claim 1 further comprising a stent attached to the thin metallic film.

12. The medical device of claim 11 wherein the stent is attached to the thin metallic film by adhesion.

13. The medical device of claim 11 wherein the adhesion comprises use of a binder.

14. The medical device of claim 11 wherein the adhesion comprises use of heat.

15. The medical device of claim 11 wherein the adhesion comprises use of a chemical bonding agent.

16. The medical device of claim 11 wherein the adhesion comprises use of a mechanical means.

17. The medical device of claim 11 wherein the attachment between the stent and the thin metallic film is achieved by a radial force exerted by the stent along the interior surface of the thin metallic film tube.

18. The medical device of claim 11 wherein the stent comprises at least one hoop structure extending between the stents proximal and distal ends.

19. The medical device of claim 17 wherein the hoop structure comprises a plurality of longitudinally arranged strut members and a plurality of loop members connecting adjacent struts.

20. A medical device for occluding a body vessel comprising:
a thin film tube capable of being longitudinally stretched by the application of mechanical energy to achieve a smaller circumferential profile, and self-expand to the pre-stretched length and diameter upon release of the mechanical energy; and
a plurality of apertures incised in the tube wall such the apertures to assist the thin film tube to longitudinally stretch.

21. A medical device for occluding a body vessel comprising:
a thin film tube capable of being longitudinally stretched by the application of mechanical energy to achieve a smaller circumferential profile, and self-expand to the pre-stretched length and diameter upon release of the mechanical energy; and
a stent attached to the interior surface of the thin metallic film.
